(19) United States
(12) Patent Application Publication
(10) Pub. No.: US 2025/0268704 A1
(43) Pub. Date: Aug. 28, 2025

(54) ZIP-TIE SYSTEMS FOR LIGAMENT AND TENDON REPAIR AND REPLACEMENT

(71) Applicant: TAS Medical, Inc., San Carlos, CA (US)

(72) Inventor: Albert K. Chin, Palo Alto, CA (US)

(21) Appl. No.: 19/040,712

(22) Filed: Jan. 29, 2025

Related U.S. Application Data
(60) Provisional application No. 63/627,063, filed on Jan. 31, 2024.

(51) Int. Cl. A61F 2/08

(52) U.S. Cl. CPC A61F 2/0811; A61F 2002/0817; A61F 2002/0847; A61F 2002/0876; A61F 2220/0008; A61F 2220/0025

(57) ABSTRACT

Multiple systems are described for repairing or replacing anatomic ligaments and tendons utilizing toothed straps and bony anchors incorporating rotatable discs that lock the straps in an axial fashion. Specialized instrumentation is used to prepare the bone and to insert the anchor into the bone while providing proper orientation of the locking disc for accepting the toothed strap.

### INCORPORATION BY REFERENCE TO ANY PRIORITY APPLICATIONS

This application claims benefit of priority to U.S. Provisional Application No. 63/627,063, filed Jan. 31, 2024, which is expressly incorporated by reference in its entirety for all purposes.

### BACKGROUND

### Field

The field of the present disclosure corresponds to repair and replacement of anatomic ligaments and tendons, more particularly repair using a self-locking strap.

### Description of the Related Art

Ligaments are fibrous connective tissue that connect two bones, while tendons are fibrous connective tissue that connect muscle to bone. Surgical repair of detached tendons is typically performed using attachment of sutures to the disrupted tendon and fixation of the attached sutures to bone with the insertion of one or more bony anchors. Disrupted ligaments may be replaced by harvesting part of the patient's own tendon as an autologous graft, or by the use of a cadaveric donor graft fixated to the origin and insertion sites by bone anchors. Tendon repair using fixation of the suture to the bone using a bony anchor may be limited by the tensile strength of the repair, even with specialized braid reinforced suture or strands composed of multiple suture loops. Furthermore, the multiple suture loops may be cumbersome and complicated to manipulate during surgery.

A ligament or tendon repair technique and instrumentation is desired that provides re-attachment and, which can be implemented in an intuitive, minimally invasive fashion.

### SUMMARY

Several systems are described herein, which are useful for repairing or replacing anatomic ligaments or tendons utilizing toothed straps and bony anchors incorporating rotatable discs that lock the straps in an axial fashion. Specialized instrumentation is also described herein, which are used to prepare the bone and to insert the anchor into the bone while providing proper orientation of the locking disc for accepting the toothed strap. Preferred alternatives are set forth below.
1. A system for repairing a tendon, the system comprising:
a self-locking strap comprising:
   one or more leaders, the one or more leaders each comprising a needle at a distal end;
   a strap portion;
a bone anchor configured for implantation into bone of a patient, the bone anchor comprising a ratchet configured to allow passage of the strap portion in a first direction and to prevent passage of the strap portion in a second direction opposite the first direction.
2. The system of alternative 1, wherein the strap portion comprises a plurality of teeth, wherein the ratchet is configured to engage the plurality of teeth.
3. The system of alternative 1 or 2, wherein the bone anchor comprises a threaded portion, wherein the ratchet is configured to rotate with respect to the threaded portion.
4. The system of any one of alternatives 1 to 3, wherein the self-locking strap comprises a plurality of leaders.
5. The system of alternative 4, wherein the self-locking strap comprises two leaders.
6. The system of any one of alternatives 1 to 5, wherein the needles of the leaders are curved needles.
7. The system of any one of alternatives 1 to 6, comprising an anchor insertion device configured to secure the bone anchor to bone of the patient.
8. The system of alternative 7, wherein the bone anchor comprises a plurality of cavities configured to mate with a plurality of pins of the anchor insertion device.
9. The system of alternative 7 or 8, wherein the anchor insertion device comprises a handle and a beam configured to be inserted into a central opening of the bone anchor.
10. The system of any one of alternatives 7 to 9, wherein the anchor insertion device comprises a sleeve configured to hold the bone anchor to the anchor insertion device prior to securing the bone anchor to the patient's bone.
11. The system of any one of alternatives 1 to 10, comprising a canulated drill configured to create a guide hole, wherein the bone anchor is configured to secure within the guide hole.
12. The system of alternative 11, wherein the canulated drill comprises a stop configured to prevent or inhibit drilling beyond a predetermined depth.
13. The system of any one of alternatives 1 to 12, wherein the system is adapted for repair of an Achilles tendon of the patient, and wherein the bone of the patient is the calcaneus bone.
14. A method for repairing a tendon, the method comprising:
securing a bone anchor to bone of a patient;
providing a self-locking strap comprising one or more leaders, the one or more leaders each comprising a needle at a distal end, and a strap portion;
securing, using the one or more leaders, the self-locking strap to a tendon of the patient;
inserting the self-locking strap into the bone anchor; and
tightening the self-locking strap.
15. The method of alternative 14, comprising cutting the self-locking strap to eliminate overhang past the bone anchor.
16. The method of alternative 14 or 15, wherein securing the self-locking strap to the tendon comprises stitching, using the one or more leaders and needles, the self-locking strap to the tendon.
17. The method of alternative 16, wherein the stitching is a locking stitch.
18. The method of any one of alternatives 14 to 17, wherein the tendon is an Achilles tendon.
19. The method of any one of alternatives 14 to 18, wherein the strap portion comprises a plurality of teeth, wherein a rachet of the bone anchor is configured to engage the plurality of teeth.
20. The method of any one of alternatives 14 to 19, wherein the bone anchor comprises a threaded portion and a ratchet, wherein the ratchet is configured to rotate with respect to the threaded portion.
21. The method of any one of alternatives 14 to 20, wherein the self-locking strap comprises a plurality of leaders.
22. The method of alternative 21, wherein the self-locking strap comprises two leaders.
23. The method of any one of alternatives 14 to 22, wherein the needles of the leaders are curved needles.
24. The method of any one of alternatives 14 to 23, wherein the securing the bone anchor to bone of a patient is conducted using an anchor insertion device.
25. The method of any one of alternatives 14 to 24, comprising cutting the one or more leaders to remove the needle at the distal end.
26. The method of any one of alternatives 14 to 25, comprising drilling a guide hole for the bone anchor.
27. The method of any one of alternatives 14 to 26, wherein the tendon is an Achilles tendon of the patient.
28. A system for replacing a ligament, the system comprising:
a self-locking strap comprising a plurality of teeth;
a first bone anchor; and
a second bone anchor, wherein each of the first bone anchor and second bone anchor are configured for implantation into bone of a patient, and wherein each of the first bone anchor and second bone anchor comprise a ratchet configured to engage the plurality of teeth to allow passage of the self-locking strap in a first direction and to prevent passage of the self-locking strap in a second direction opposite the first direction.
29. The system of alternative 28, wherein the self-locking strap comprises a first portion and a second portion, wherein the teeth of the first portion are oriented opposite the teeth of the second portion.
30. The system of alternative 28 or 29, wherein, when assembled at a joint of the patient, the first bone anchor, the second bone anchor, and the self-locking strap are configured to prevent or inhibit separation of the joint.
31. The system of any one of alternatives 28 to 30, wherein each of the first bone anchor and the second bone anchor comprises a threaded portion, wherein the ratchet is configured to rotate with respect to the threaded portion.
32. The system of any one of alternatives 28 to 31, comprising an anchor insertion device configured to secure the bone anchor to bone of the patient.
33. The system of alternative 32, wherein the bone anchor comprises a plurality of cavities configured to mate with a plurality of pins of the anchor insertion device.
34. The system of alternative 32 or 33,wherein the anchor insertion device comprises a handle and a beam, wherein the beam is configured to be inserted into a central opening of the bone anchor.
35. The system of any one of alternatives 32 to 34, wherein the anchor insertion device comprises a sleeve configured to hold the bone anchor to the anchor insertion device prior to securing the bone anchor to the patient's bone.
36. The system of any one of alternatives 28 to 35, comprising a canulated drill configured to create a guide hole, wherein the bone anchor is configured to secure within the guide hole.
37. The system of alternative 36, wherein the canulated drill comprises a stop configured to prevent or inhibit drilling beyond a predetermined depth.
38. The system of any one of alternatives 28 to 37, wherein the system is adapted for repair of a knee ligament of the patient.
39. A method for replacing a ligament, the method comprising:
securing a first bone anchor to a first bone of a patient;
securing a second bone anchor to a second bone of the patient;
providing a self-locking strap comprising a plurality of teeth;
inserting the self-locking strap into the first bone anchor and second bone anchor; and
tightening the self-locking strap against the first bone anchor or second bone anchor.
40. The method of alternative 39, comprising cutting the self-locking strap to eliminate overhang past the first bone anchor and second bone anchor.
41. The method of alternative 39 or 40, wherein the self-locking strap comprises a first portion and a second portion, wherein the teeth of the first portion are oriented opposite the teeth of the second portion.
42. The method of any one of alternatives 39 to 41, wherein the first bone anchor and second bone anchor each comprises a ratchet and a threaded portion, wherein the ratchet is configured to rotate with respect to the threaded portion, and wherein the ratchet is configured to engage the plurality of teeth of the self-locking strap.
43. The method of any one of alternatives 39 to 42, comprising drilling a first guide hole for the first bone anchor and a second guide hole for the second bone anchor.
44. The method of any one of alternatives 39 to 43, wherein the securing the first bone anchor to a first bone of a patient is conducted using an anchor insertion device.
45. The method of any one of alternatives 39 to 44, wherein the securing the second bone anchor to a second bone of a patient is conducted using an anchor insertion device.
46. The method of any one of alternatives 39 to 45, wherein the ligament is a ligament of the patient's knee, wherein the first bone is one of the femur or tibia and wherein the second bone is the other of the femur or tibia.

### BRIEF DESCRIPTION OF THE DRAWINGS

FIGS. 1A-1C depict the bone anchor and the configuration of its components.

FIG. 2 illustrates the bone anchor insertion device.

FIG. 3 illustrates the bone anchor loaded onto the bone anchor insertion device.

FIG. 4. shows the single bone anchor version of a toothed strap containing sutures comprising curved needles.

FIG. 5 shows the double bone anchor version of toothed strap containing teeth on opposite sides of the strap.

FIG. 6A-6C depict a sequence of steps, which can be used to insert the bone anchor.

FIG. 7A-7D illustrates a sequence of steps, which can be used in Achilles tendon repair.

FIG. 8A-8D illustrates a sequence of steps, which can be used in medial collateral ligament replacement.

### DETAILED DESCRIPTION

The present disclosure provides various configurations of self-locking straps, e.g., polymeric straps, suitable for tendon and/or ligament repair or replacement, as well as instrumentation configured to place or secure these implants into soft tissue and bone. A bone anchor, for example, can include a large, coarse pitched thread, and a tapering distal tip. The head of the bone anchor can include a housing that accommodates a freely rotating disc with a central ratchet locking mechanism to receive and lock a self-locking strap. The anterior face of the bone anchor can include two or more small cavities spaced around its perimeter that accept matching pins on the anchor insertion device. The anchor insertion device can include a handle incorporating a rotating knob with small diameter pins protruding from the distal end of the knob used to screw the anchor into the bone. A thin beam can be rigidly fixed to the handle and can extend through an inner cavity of the knob and at a short distance past the distal ends of the pins. The thin beam can insert into the ratchet mechanism in the rotatable disc and it can be suitable to maintain the ratchet mechanism in the correct position so as to accept and lock the toothed strap while the anchor is rotated into locking position in the bone. An elastic sleeve may extend from the anchor insertion device onto the proximal portion of the bone anchor housing to hold the bone anchor assembly on the anchor insertion device without it falling off the tool during bone insertion. Prior to anchor insertion, the insertion site in the bone can be prepared by drilling and tapping matching threads to accept the anchor. In some embodiments, a countersink may be applied to the bone as needed to accommodate an anchor head with an outer diameter in excess of the major thread diameter.

At least two versions of the self-locking strap may be used with the bone anchor. One exemplary version (a single bone anchor version) allows proximal soft tissue attachment and distal bone anchoring, while an exemplary second version allows attachment to bone anchors placed in the proximal and distal positions. The single-bone anchor version comprises a polymer strap having teeth extending, preferably or optionally, the full length of the strap and two monofilament sutures, preferably or optionally, having curved needles extending from the corners of the proximal end of the strap. The proximal end of the strap is attached to soft tissue such as tendon, muscle or fascia. Following placement of the bone anchor at the distal insertion site, the distal portion of the strap can be cut to the correct length for insertion into the locking disc in the head of the anchor. The double bone anchor version comprises a polymer strap containing a short length (e.g., approximately 2-3 cm) of teeth on one end of the strap, followed by a full length of teeth on the opposite side of the strap. The end of the strap containing a short length of teeth can be inserted into the proximal bone anchor, followed by transection of the opposite end of the strap to the proper length for insertion into the distal bone anchor. The self-locking strap of both versions may be made of metal or, preferably, a high tensile strength polymer such as nylon, polyetherimide (Ultem), polyether ether ketone (PEEK), or polyamideimide (PAI).

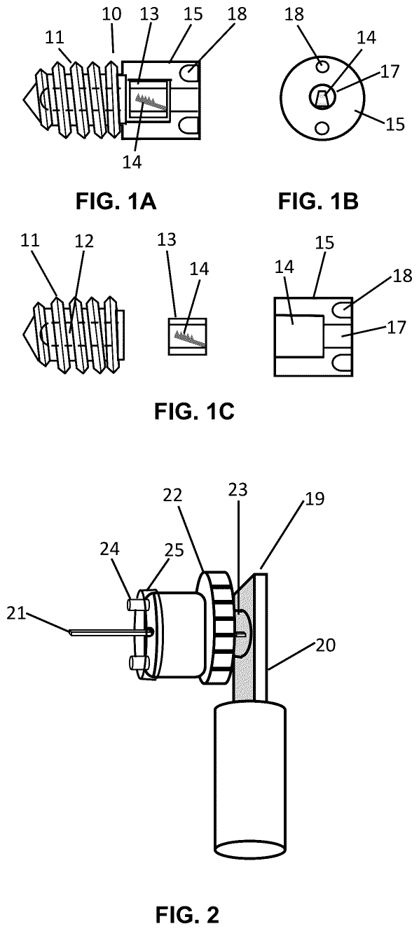

Now with reference to FIG. 1A, which illustrates the assembled bone anchor 10, having a distal threaded section 11 (e.g., having coarse threads), a proximal cap 15, and a freely rotating locking disc 13 constrained by the proximal cap 15 (shown in a cross-sectional view in FIG. 1A). The locking disc 13 has a central cavity that houses a ratchet mechanism 14 (e.g., pawls). The proximal face of the proximal cap 15 contains multiple small cavities 18 that interface with driving pins on the insertion device, which is described below. The distal threaded section 11 of the anchor 10 may be made of a metal such as stainless steel or titanium, or a polymer such as nylon, polyether ether ketone (PEEK), polyetherimide (Ultem), or polyamideimide (PAI) for example. In other embodiments, the distal threaded section 11 may be a bioabsorbable material such as Poly-L-Lactic Acid (PLLA). The proximal cap 15 may be made of a polymer, or it may be a metal such as stainless steel. The locking disc 13 may be made of a non-bioabsorbable polymer, metal, or a combination of polymer and metal. FIG. 1B is an end-view of the bone anchor 10, with cavities 18 spaced around the periphery of the proximal cap 15, and a central opening 17 through which the rachet mechanism 14 may be observed. While the present embodiment of the proximal cap 15 has two cavities 18, there may be more cavities in some embodiments. FIG. 1C is an exploded view showing the three components that form bone anchor 10, including the distal threaded section 11 and a central cavity 12, the locking disc 13, and the proximal cap 15. In some embodiments, cap 15 may be attached to threaded section 11, which can maintain the locking disc 13 in a fixed or captive position.

FIG. 2 shows the anchor insertion device 19 having a handle 20 containing a rigidly attached thin beam 21. Knob 22 is attached to the handle 20 via a connector 23 that allows knob 22 to rotate freely with respect to the handle 20. The beam 21 resides in an open central channel that extends the length of knob 22 and beyond its distal end. Multiple pins 24 protrude from the distal face of knob 22 to interface with the implantable bone anchor 10. An elastic sleeve 25 extends forward from the distal aspect of knob 22 to hold the bone anchor 10 on the anchor insertion device 19 before and during anchor insertion into bone. The handle 20, beam 21, and pins 24 may be made of a metal, such as stainless steel, or a polymer, such as Nylon or PEEK. The handle 10, beam 21, and pins 24 are not necessarily made of the same materials.

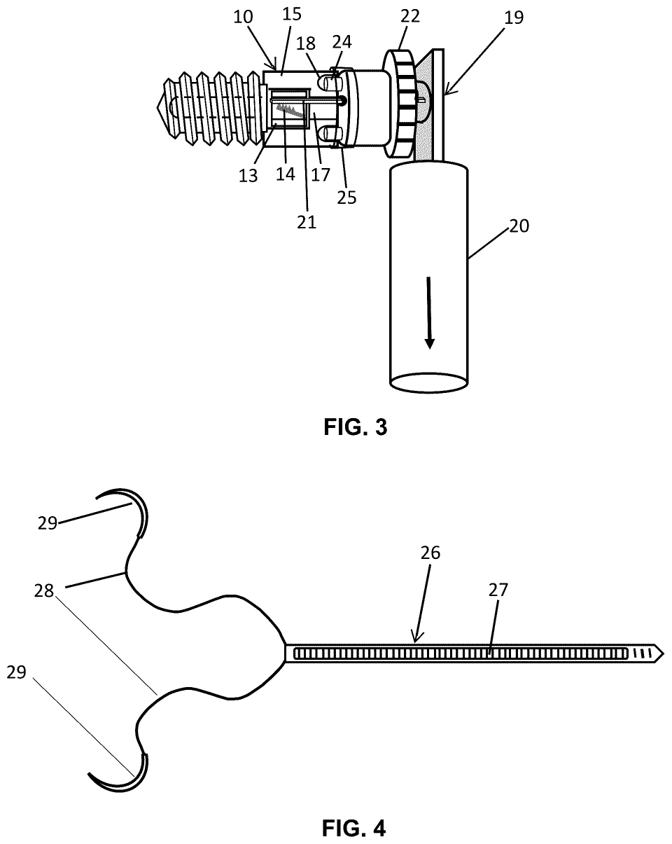

FIG. 3 illustrates the bone anchor 10 loaded onto the anchor insertion device 19. The pins 24 have an outer diameter that is a sliding fit with the cavities 18 in the proximal cap 15 of bone anchor 10, and the elastic sleeve 25 extends onto the proximal cap 15 to hold the bone anchor 10 in position during insertion into bone. The beam 21 extends through the opening 17 in the proximal cap 15 of bone anchor 10, and continues through the central cavity of the locking disc 13 superior to the ratchet mechanism 14. The beam 21 prevents the locking disc 13 from rotating, thus maintaining its orientation while the bone anchor 10 is rotatably threaded into bone by the surgeon rotating the knob 22. When the handle 20 of the anchor insertion device 19 is directed inferiorly along the axis of the tendon or ligament repair toothed strap, the locking disc 13 will be oriented properly to accept and lock the toothed strap.

FIG. 4 depicts self-locking strap 26 for tendon repair. The self-locking strap 26 contains teeth 27 that extend the full length on one side of strap 26. Two leaders 28 are attached to the proximal end of strap 26, and each leader 28 has at its distal end a curved needle 29. The leaders 28 can be sutures or a mesh material.

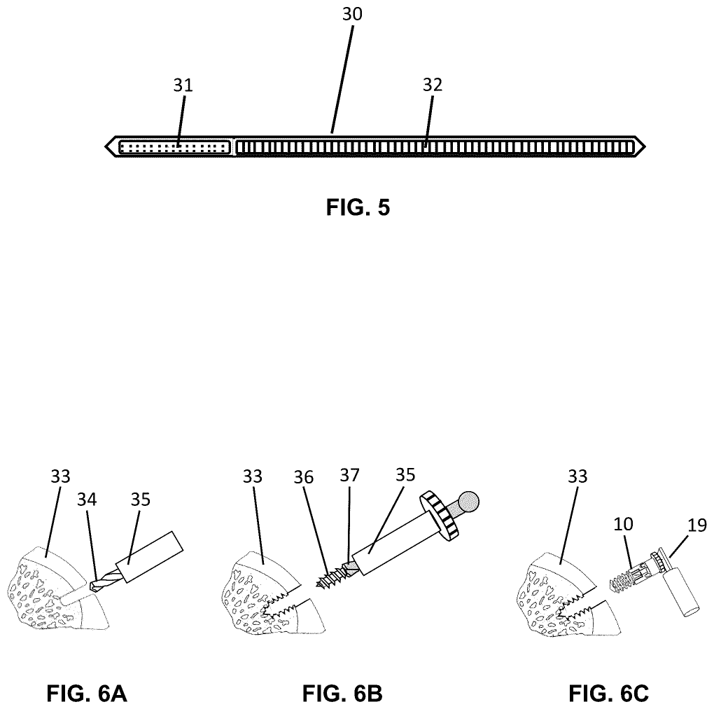

FIG. 5 depicts a self-locking strap 30 for ligament repair, which contains a first length of teeth 31 on one end of the strap 30, followed by a second length of teeth 32 on the opposite side of the strap 30. In some examples, the first length of teeth 31 and the second length of teeth 32 may be approximately equal, or the first length of teeth 31 may be shorter than the second length of teeth 32, or vice versa. The teeth of the first length of teeth 31 can be oriented in an opposite direction relative to the teeth of the second length of teeth 32. That is, the direction of the ramping of the teeth may be in the opposite direction.

FIG. 6A illustrates the first step in bone anchor placement, consisting of drilling a guide hole in the bone 33. The guide hole can include a length equal to the length of the bone anchor. A drill bit 34 may contain a stop 35 to prevent drilling of a predetermined, excessive depth of hole formation. FIG. 6B shows the next step of applying a tap 36 to form threads in the bone 33 and optionally forming a countersink in the tapped hole. A specialized tool comprised of a distal tap 36 combined with a cannulated drill 37 and a drill stop 35 may be used to perform this step. FIG. 6C shows insertion of the anchor 10 into the bone 33 using the insertion device 19.

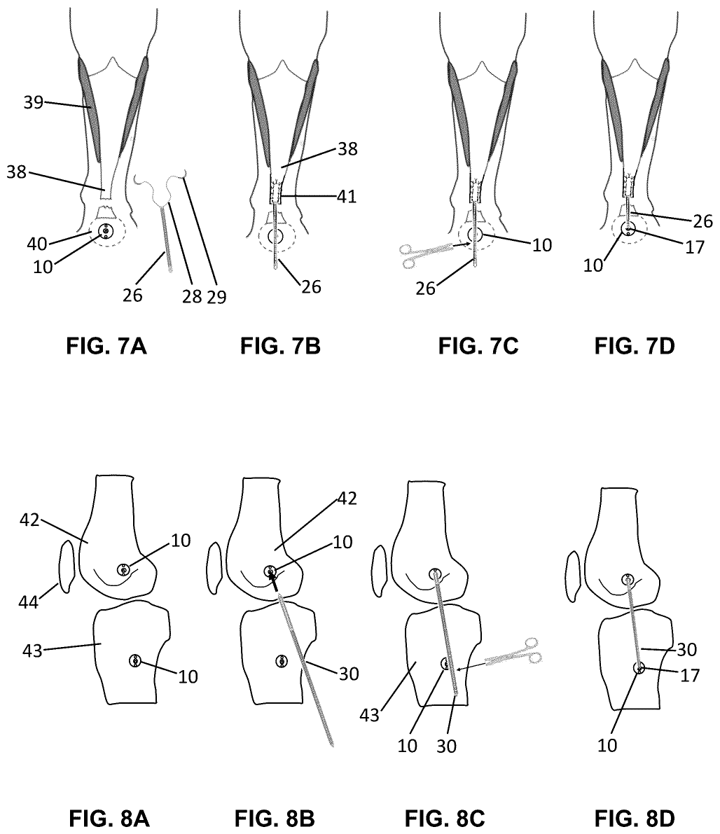

FIG. 7A shows the posterior aspect of the lower leg, with the ruptured Achilles tendon 38 lying central to the gastrocnemius muscle 39. A bone anchor 10 has been implanted in the calcaneus bone 40 in the patient's heel, and a self-locking strap 26 including leaders 28 comprising curved needles 29 is brought into position for placement on the proximal portion of the ruptured Achilles tendon 38. FIG. 7B shows that a Krakow type of locking stitch 41 has been applied (using the leaders 28) to attach the self-locking strap 26 to the proximal portion of the ruptured Achilles tendon 38. FIG. 7C shows the attached self-locking strap 26 being formed to the proper length by cutting it at or near the inferior perimeter of the bone anchor 10. The cut end of the self-locking strap 26 is inserted into the opening 17 in the bone anchor 10 to lock it into place via the ratchet mechanism 14 (FIG. 1A) to complete the repair. It should be understood that, though FIGS. 7A-7D are discussed with reference to Achilles tendon repair, the devices and methods discussed can be suitably applied to repair other damaged tendons and/or joints.

FIG. 8A shows the medial aspect of the knee, with a bone anchor 10 inserted into the medial aspect of the distal femur 42 and a second bone anchor 10 inserted into the medial aspect of the proximal tibia 43. The patella 44 is observed in the anterior aspect of the knee. FIG. 8B illustrates the short length of self-locking strap 30 inserted into bone anchor 10 in the medial aspect of femur 41. As discussed with reference to FIG. 5, the short length of teeth and the long length of teeth of the ligament self-locking strap can each include teeth oriented in the opposite direction so that ratchets of each of the bone anchors 10 can prevent or inhibit motion of the distal femur 42 away from the proximal tibia 43. In such an example, the ratchets of the bone screws can be oriented in opposite directions. FIG. 8C shows the ligament self-locking strap 30 being formed to the proper length by cutting it at the inferior perimeter of the distal bone anchor 10 in the medial aspect of tibia 43. FIG. 8D shows the cut distal end of ligament self-locking strap 30 inserted into the opening 17 in the proximal tibial bone anchor 10 to lock it into place to complete the medial collateral ligament repair. It should be understood that, though FIGS. 8A-8D are discussed with reference to a knee joint, the devices and methods discussed can be suitably applied to other joints and/or ligaments.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended alternatives and equivalents thereto, inventive embodiments may be practiced otherwise than as specifically described. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system for repairing a tendon, the system comprising:
a self-locking strap comprising:
one or more leaders, the one or more leaders each comprising a needle at a distal end;
a strap portion;
a bone anchor configured for implantation into bone of a patient, the bone anchor comprising a ratchet configured to allow passage of the strap portion in a first direction and to prevent passage of the strap portion in a second direction opposite the first direction.

2. The system of claim 1, wherein the strap portion comprises a plurality of teeth, wherein the ratchet is configured to engage the plurality of teeth.

3. The system of claim 1, wherein the bone anchor comprises a threaded portion, wherein the ratchet is configured to rotate with respect to the threaded portion.

4. The system of claim 1, wherein the self-locking strap comprises a plurality of leaders.

5. The system of claim 4, wherein the self-locking strap comprises two leaders.

6. (canceled)

7. The system of claim 1, comprising an anchor insertion device configured to secure the bone anchor to bone of the patient.

8. The system of claim 7, wherein the bone anchor comprises a plurality of cavities configured to mate with a plurality of pins of the anchor insertion device.

9. The system of claim 7, wherein the anchor insertion device comprises a handle and a beam configured to be inserted into a central opening of the bone anchor.

10. The system of claim 7, wherein the anchor insertion device comprises a sleeve configured to hold the bone anchor to the anchor insertion device prior to securing the bone anchor to the patient's bone.

11. (canceled)

12. (canceled)

13. (canceled)

14. A method for repairing a tendon, the method comprising:
securing a bone anchor to bone of a patient;
providing a self-locking strap comprising one or more leaders, the one or more leaders each comprising a needle at a distal end, and a strap portion;
securing, using the one or more leaders, the self-locking strap to a tendon of the patient;
inserting the self-locking strap into the bone anchor; and
tightening the self-locking strap.

15. (canceled)

16. The method of claim 14, wherein securing the self-locking strap to the tendon comprises stitching, using the one or more leaders and needles, the self-locking strap to the tendon.

17. (canceled)

18. The method of claim 14, wherein the tendon is an Achilles tendon.

19. The method of claim 14, wherein the strap portion comprises a plurality of teeth, wherein a rachet of the bone anchor is configured to engage the plurality of teeth.

20. (canceled)

21. The method of claim 14, wherein the self-locking strap comprises a plurality of leaders.

22. The method of claim 21, wherein the self-locking strap comprises two leaders.

23. The method of claim 14, wherein the needles of the leaders are curved needles.

24. The method of claim 14, wherein the securing the bone anchor to bone of a patient is conducted using an anchor insertion device.

25. (canceled)

26. The method of claim 14, comprising drilling a guide hole for the bone anchor.

27. The method of claim 14, wherein the tendon is an Achilles tendon of the patient.

28. A system for replacing a ligament, the system comprising:
a self-locking strap comprising a plurality of teeth;
a first bone anchor; and
a second bone anchor, wherein each of the first bone anchor and second bone anchor are configured for implantation into bone of a patient, and wherein each of the first bone anchor and second bone anchor comprise a ratchet configured to engage the plurality of teeth to allow passage of the self-locking strap in a first direction and to prevent passage of the self-locking strap in a second direction opposite the first direction.

29. (canceled)
